(19) United States
(12) Patent Application Publication
(10) Pub. No.: US 2017/0281274 A1
(43) Pub. Date: Oct. 5, 2017

(54) DEVICE AND METHOD FOR TREATING A SKIN LESION

(71) Applicant: Hector Luis Santana, JR., Mesa, AZ (US)

(72) Inventor: Hector Luis Santana, JR., Mesa, AZ (US)

(21) Appl. No.: 15/478,120

(22) Filed: Apr. 3, 2017

Related U.S. Application Data
(60) Provisional application No. 62/318,051, filed on Apr. 4, 2016.

(51) Int. Cl. A61B 18/20

(52) U.S. Cl. CPC A61B 18/203; A61B 18/20; A61B 18/201; A61B 2018/00452

(57) ABSTRACT

An electronic device for treating a skin lesion by destroying an affected skin cell using an electric current includes a hollow cylindrical housing with a female casing configured to mate with a male casing via a threading. Further, a printed circuit board is housed within the hollow cylindrical housing and is configured to generate electric current by using a battery. Furthermore, a needle is attached to a dispersive electrode of the printed circuit board for carrying the electric current from the electronic device to the affected skin cell in order to destroy the skin lesion. The electronic device is used by inserting the battery in the clip attached to the printed circuit board, pressing the button to actuate circuit, contacting the skin lesion with the needle and then passing the electric current via the needle for a specified period.

### RELATED APPLICATION

This application claims priority to provisional patent application U.S. Ser. No. 62/318,051 filed on Apr. 4, 2016, the entire contents of which is herein incorporated by reference.

### BACKGROUND

The embodiments herein relate generally to an electronic device and a method for treating a skin lesion, such as a wart and a skin growth. Prior to this invention, the treatment options included expensive, painful and inconvenient procedures such as electrodessication, surgical curettage and cryosurgery. Further, the aforementioned treatments required a skilled medical practitioner, such as a dermatologist, to perform the procedures. The present invention solves the problem.

### SUMMARY

An electronic device for treating a skin lesion by destroying an affected skin cell using an electric current includes a hollow cylindrical housing with a female casing configured to mate with a male casing via a threading. Further, a printed circuit board is housed within the hollow cylindrical housing and is configured to generate electric current by using a battery. Furthermore, a needle is attached to a dispersive electrode of the printed circuit board for carrying the electric current from the electronic device to the affected skin cell in order to destroy the skin lesion. The electronic device is used by inserting the battery in the clip attached to the printed circuit board, pressing the button to actuate circuit, contacting the skin lesion with the needle and then passing the electric current via the needle for a specified period.

### BRIEF DESCRIPTION OF THE FIGURES

The detailed description of some embodiments of the invention is made below with reference to the accompanying figures, wherein like numerals represent corresponding parts of the figures.

FIG. 1 shows a perspective view of one embodiment of the present invention;

FIG. 2 shows an exploded view of one embodiment of the present invention;

FIG. 3 is a section view of one embodiment of the present invention taken along line 3-3 in FIG. 1;

FIG. 4 is a schematic view of one embodiment of the present invention; and

FIG. 5 is a schematic view of one embodiment of the present invention.

### DETAILED DESCRIPTION OF CERTAIN EMBODIMENTS

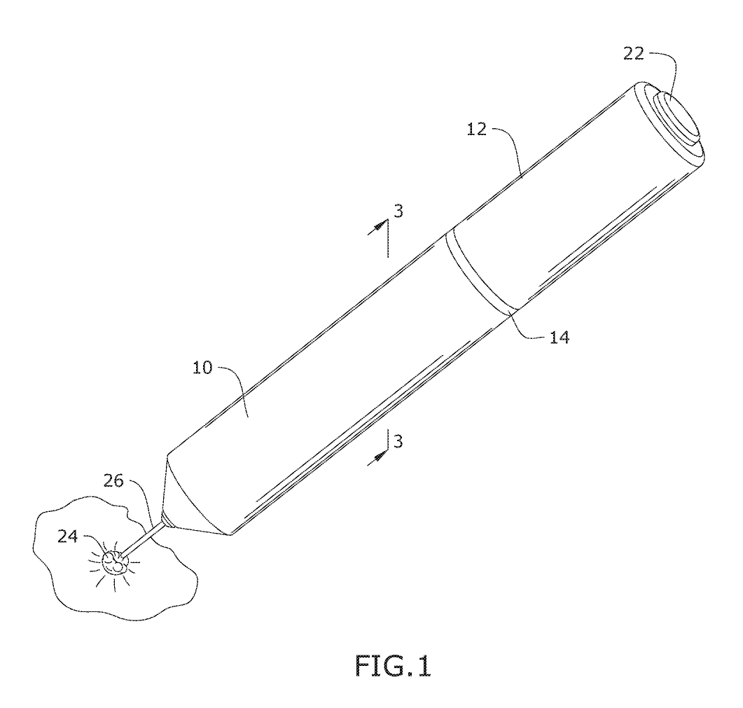

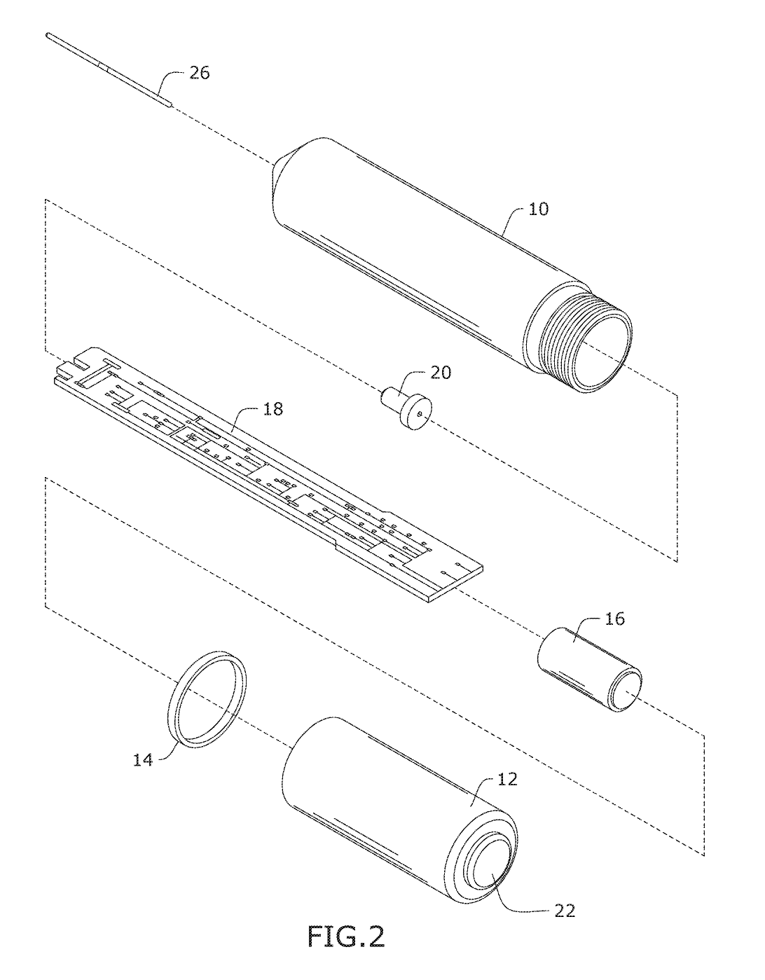

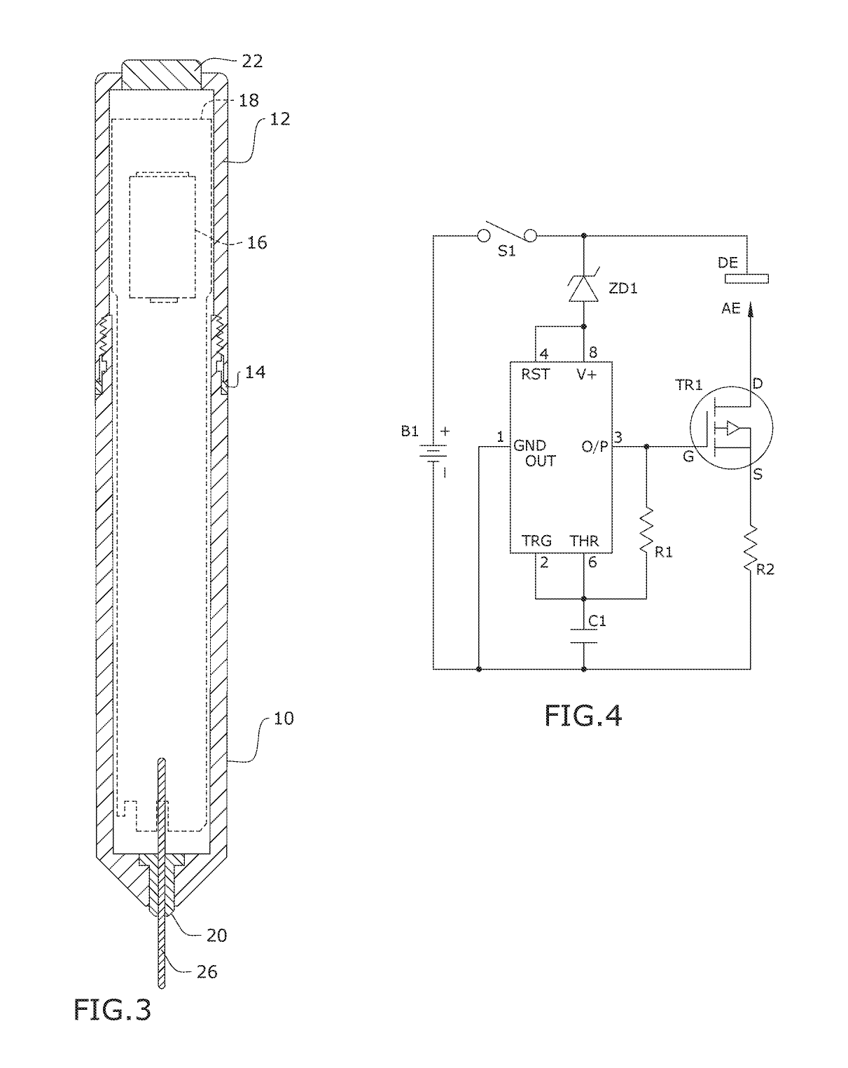

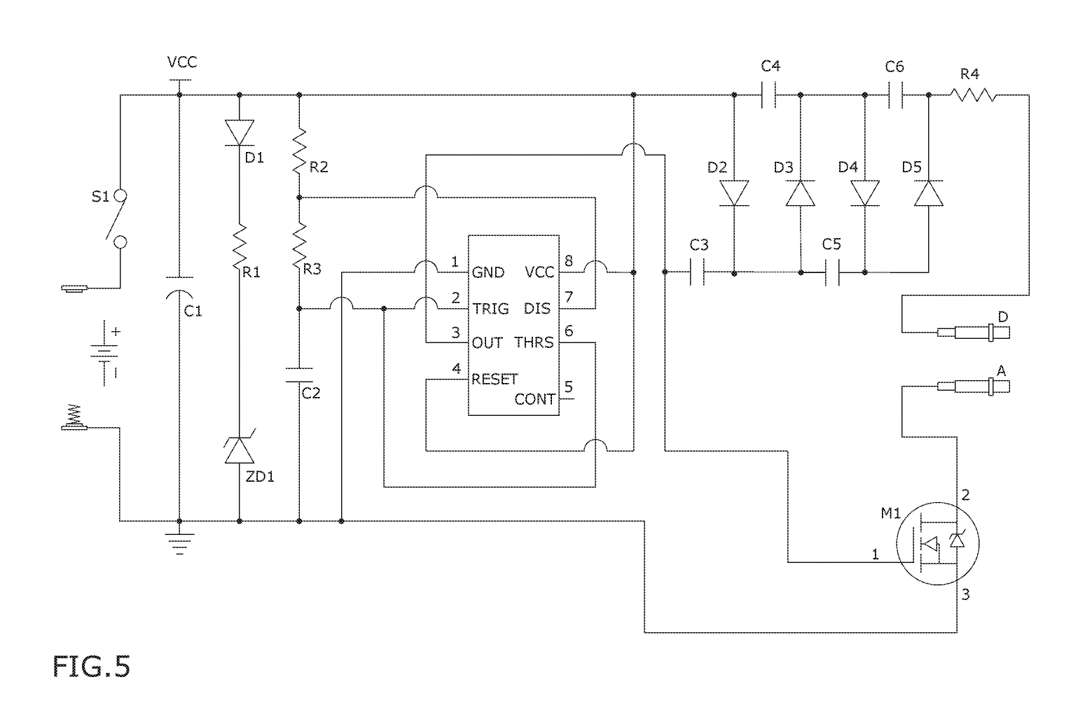

By way of example, and referring to FIG. 1-5, one embodiment of the present system comprises an electronic device 10 for treating a skin lesion L, such as a wart and a skin growth, by destroying an affected skin cell using an electric current. The electronic device further comprises a hollow cylindrical housing 14, having a female casing 16 configured to mate with a male casing 18 via a threading 20 and formed an airtight seal by inserting a seal ring 22 and a black rubber ring 24 in between the female casing and the male casing. A printed circuit board 26 is inserted within the hollow cylindrical housing and is configured to generate the electric current as well as to provide a visual indicator.

The printed circuit board 26 is mechanically coupled to a collar 28. The collar 28 is further electrically coupled to a needle 30. The printed circuit board 26 is mechanically coupled to a cathode 32 parallel to the needle 30.

In one embodiment, the printed circuit board 26 further comprises an integrated circuit 34 electrically coupled to the battery 36 with a switch 38. A positive end of battery 36 is electrically coupled to a first pin 40 and the integrated circuit 34. A negative end of battery 36 is electrically coupled to a second pin 42 and the integrated circuit 34 with a MOSFET 44. A light emitting diode 46 is electrically coupled to the first pin 40 in order to indicate when the circuit is active. In another embodiment, a plurality of capacitors in parallel 48 can be electrically coupled to the first pin 40 in order to expand the ability of the circuit to handle greater voltage and current.

A method for treating a skin lesion by destroying an affected skin cell using an electric current from the electronic device comprises: inserting the battery in the clip attached to the printed circuit board, pressing the button to actuate circuit, contacting the skin lesion with the needle and passing the electric current via the needle for a specified period. The specified period is between 3 and 5 minutes of contact of the needle with the skin lesion when the electronic device is actuated.

The hollow cylindrical housing comprising the female casing and the male casing can be made of light-weight metal, such as aluminum. Further, the aluminum can be configured to provide a grip by using sanded surface. The seal ring can be made of metal with glazed surface in order for a consumer to identify the female casing and the male casing. The seal ring can be a brass O-ring.

The electronic device can be modified to exclude the visual indicator. On the other hand, the electronic device can be improved to include a variable setting to enable the consumer with an option to change output of the electric current. Further, the electronic device can be configured to include the battery with 24 V instead of 12 V. Furthermore, the electronic device can be configured to include an ICM7555 chip. The electronic device can be modified to treat sun spots, freckles and to permanently remove hair.

As used in this application, the term “a” or “an” means “at least one” or “one or more.”

As used in this application, the term “about” or “approximately” refers to a range of values within plus or minus 10% of the specified number.

As used in this application, the term “substantially” means that the actual value is within about 10% of the actual desired value, particularly within about 5% of the actual desired value and especially within about 1% of the actual desired value of any variable, element or limit set forth herein.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Any element in a claim that does not explicitly state “means for” performing a specified function, or “step for” performing a specified function, is not to be interpreted as a “means” or “step” clause as specified in 35 U.S.C. §112, ¶6. In particular, any use of “step of” in the claims is not intended to invoke the provision of 35 U.S.C. §112, ¶6.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. An electronic device configured to treat a skin lesion by destroying an affected skin cell using an electric current, the electronic device further comprising:
a hollow cylindrical housing;
a printed circuit board, housed inside the hollow cylindrical housing,
a battery, inserted in a clip attached to the printed circuit board to power the electronic device;
a needle, encircled by a collar and attached to a first pin on the printed circuit board for carrying the electric current from the electronic device to the affected skin cell to destroy the skin lesion.

2. The electronic device of claim 1, wherein the hollow cylindrical housing further comprises: a female casing configured to mate with a male casing via a threading and formed an airtight seal by inserting a seal ring and a black rubber ring in between the female casing and the male casing.

3. The electronic device of claim 2, wherein the printed circuit board further comprises: wherein the printed circuit board further comprises an integrated circuit electrically coupled to the battery with a switch.

4. The electronic device of claim 3, wherein a positive end of the battery is electrically coupled to the first pin and the integrated circuit.

5. The electronic device of claim 4, wherein a negative end of the battery is electrically coupled to a second pin and the integrated circuit with a MOSFET.

6. The electronic device of claim 5, wherein A light emitting diode is electrically coupled to the first pin in order to indicate when the printed circuit board is active.
